(19) United States
(12) Patent Application Publication
(10) Pub. No.: US 2026/0117398 A1
(43) Pub. Date: Apr. 30, 2026

(54) Conversion of carbon dioxide to ethyl formate with low GHG emission

(71) Applicants: Bhupender Singh Minhas, Bridgewater, NJ (US); Walter Weissman, Odessa, FL (US)

(72) Inventors: Bhupender Singh Minhas, Bridgewater, NJ (US); Walter Weissman, Odessa, FL (US)

(21) Appl. No.: 18/739,180

(22) Filed: Jun. 10, 2024

(51) Int. Cl. C25B 3/26; B01J 19/18; C07C 67/08; C25B 3/07; C25B 15/08

(52) U.S. Cl. CPC C25B 3/26; B01J 19/1893; C07C 67/08; C25B 3/07; C25B 15/081

(57) ABSTRACT

The invention relates to a low greenhouse gas emission process for converting carbon dioxide to a commodity chemical, ethyl formate. Carbon dioxide is converted to an aqueous formic acid product using an electro-chemical process, wherein the concentration of formic acid is about 10 wt %. The aqueous formic acid is converted to ethyl formate by esterification of formic acid with bioethanol in a reactor. The product, ethyl formate, is separated from the reaction product and water rejected using an integral low-energy membrane separation system.

### BACKGROUND

Modern civilization's energy and transportation needs have resulted in widespread combustion of carbon based (fossil) fuels, which has increased and continues to increase carbon dioxide (CO₂) emission and its concentration in the environment. This increase of CO₂ concentration in the environment is contributing to global climate changes, with potential for significant consequences and a negative impact on humanity. Government and non-government agencies around the world have initiated programs to find ways to reduce CO₂ emissions. The US Government has set a goal of a 50 percent reduction in greenhouse gas emissions by 2030, zero emissions in the power sector by 2035 and net-zero emissions economy-wide by 2050.

One of the options being pursued is to capture CO₂ at the emission source. Several approaches are being followed as well as developed to capture CO₂ from large scale industrial sources where volume and concentration are high to make the approaches practical and economically acceptable. However, substantial CO₂ emissions come from very widely distributed smaller sources such as smaller combustion engines used in automobiles, ships, and planes. Developing an economically acceptable technology to capture CO₂ from these smaller mobile sources is more challenging. Many organizations are pursuing solutions to capture CO₂ directly from the air commonly referred to as Direct Air Capture or DAC.

The captured CO₂ can be sequestered underground deep in mines or deep underwater in the oceans. The conditioning (compression) and transport of CO₂ to these sequestration sites, however, can be an expensive proposition as well as require energy intensive processes which have their own carbon emissions. Prior art carbon sequestration often results in excessive costs and even production of more GHG than removed from the atmosphere, relying on government subsidies and/or sales of carbon credits to make these approaches appear energy and/or economically feasible.

Another option is to convert the captured CO₂ to a chemical including fuels using low GHG emission processes. The present invention is directed to processes for the efficient conversion of CO₂ to a chemical, resulting in a financial and energy beneficial means for use of CO₂.

There are several approaches being followed for conversion of CO₂ to chemicals. The Gas Technology Institute reports developing a dehydration membrane reactor for converting CO₂ to chemicals including dimethyl carbonate. Media & Process Technology reports technology developments to convert CO₂ to dimethyl carbonate. The University of Wisconsin reports an approach for conversion of CO₂ to calcium carbonate. Colorado School of Mines reports working on conversion of CO₂ to methane. Advanced Cooling Technology in collaboration with Lehigh University reports working on CO₂ reforming with Methane using plasma to produce H₂ and CO. Advanced Energy Materials reports CO₂ conversion to methanol, using plasma catalysis. CO₂, therefore, can be used to make a number of chemicals, however, most are not commercially viable from an economics or energy use perspective.

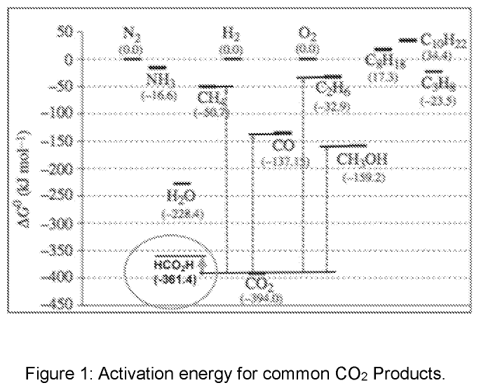

The conversion of CO₂ to form alternatives typically involves rejection of oxygen content as coproduced water. This involves production and consumption of added hydrogen resulting in a net energy input far higher than the energy content of the product. The conversion of CO₂ to formic acid of the present invention retains the COO function and thereby avoids the energy waste associated with water formation. Conversion of formic acid to ethyl formate, the target of our invention, retains the COO function avoiding this energy penalty. The advantage of formic acid as a product from CO₂ has been noted by other researchers as referenced in FIG. 1.

Typically, formic acid is produced commercially by a two-step process. In step 1, methanol reacts with carbon monoxide (˜10 atm, 80° C.) to produce methyl formate. Hydrolysis of methyl formate is conducted with a large excess of water to overcome the unfavorable thermodynamics. The product acid is then concentrated to 85 wt % plus by distillation plus extraction.

Generally, ethyl formate is produced in a single pot batch process using concentrated formic acid (85 wt %+) and ethanol as raw materials along with sulfuric acid as catalyst. In the production process water formed during esterification reaction is continuously separated as ternary azeotrope (i.e. ethyl formate, water and ethanol) vaporizing from the reactor into a column and condensed at the top of the column. The condensed liquid forms two phases. The upper layer is a mixture of ester and alcohol, which is saturated with water, and the lower layer is mainly water saturated with ester and alcohol. The upper layer is refluxed back to the reactor and the lower layer is taken off the esterification reaction.

At the end of the esterification reaction, ethyl formate is distilled off the reactor. Ethyl formate carries with it some formic acid, ethanol, and water. This product is dried using anhydrous calcium chloride. A fraction at 53 to 58 C of the dried product is taken as finished product, ethyl formate. In this process the conversion of formic acid to ethyl formate is about 80%. This traditional process requires high energy intensity, high raw material consumption, low equipment production capacity and a long production cycle.

CN1039226C describes a semi-continuous process for ethyl formate synthesis. Their process uses formic acid containing 15-16% water and ethanol containing 7-8% water. Sulfuric acid is used as a catalyst. The reactor is charged with formic acid to ethanol molar ratio of 1:2.5-5.0 along with the catalyst, sulfuric acid. In addition, formic acid and ethanol mixture in molar ratio of 1:1.0-1.1 is added dropwise in the reactor. Ethyl formate is continuously distilled off the reactor. Ethyl formate is condensed and cooled. Around 60 to 65% of the ethyl formate is refluxed back to the reactor and the rest is taken as the finished product. Ethyl formate produced meets the purity standard for perfume industry. This can be further purified by ethylene glycol extractive distillation.

### SUMMARY OF THE INVENTION

The present invention teaches an energy-efficient and cost-effective continuous process of producing ethyl formate from CO₂. In the process, CO₂ is first converted to formic acid, thermodynamically the most feasible state, using a conventional electrochemical process. Electro-chemical process uses CO₂ and water to produce aqueous formic acid in the concentration range of around 10 wt %.

Dilute formic acid in the current invention is used in an esterification reaction to react with bioethanol to synthesize ethyl formate and water in the presence of a catalyst. The reaction is conducted in a continuous flow reactor such as either a continuous flow stirred tank reactor (CFSTR), or continuous flow plug flow reactor (CFPFR). The ethyl formate as synthesized is separated from the reactor content and produced at commercial grade purity with a membrane separation process. Ethyl formate, being the lowest boiling component among all the reaction mixture components, is separated using a pervaporation membrane process.

In one embodiment of the present invention, the esterification reaction mixture is processed in a recycle mode through one or more membrane separation processes to separate ethyl formate formed in the reactor as well as to remove excess water formed during the esterification reaction.

In another embodiment, the reaction temperature is maintained high enough to be able to evaporate ethyl format as it forms in the reactor (CFSTR or CFPFR). It will carry some ethanol along with other reactor mixture components. The vapor may be condensed using a condenser, e.g., an overhead condenser. The condensed liquid is processed through a membrane system to separate ethyl formate from other components. The other components are recycled back to the reactor.

In another embodiment, the reaction is carried out in a membrane reactor where ethyl formate is separated from the reaction mixture as it forms using a pervaporation membrane separation process.

Alternate embodiments include removal of water from the feed to the reactor using a membrane system. This increases the formic acid concentration in the feed to the reactor to greater than about 10 wt %.

Alternate process configurations include membrane separation of water from the feed as well as from reactor effluent and also membrane separation of ethyl formate from the reaction mixture are contemplated under this invention. The recovery of ethyl formate as it forms during the reaction also improves conversion of formic acid to ethyl formate in the reactor.

### DETAILED DESCRIPTION OF THE INVENTION

The present invention teaches an innovative technique to convert CO₂ to a chemical, ethyl formate.

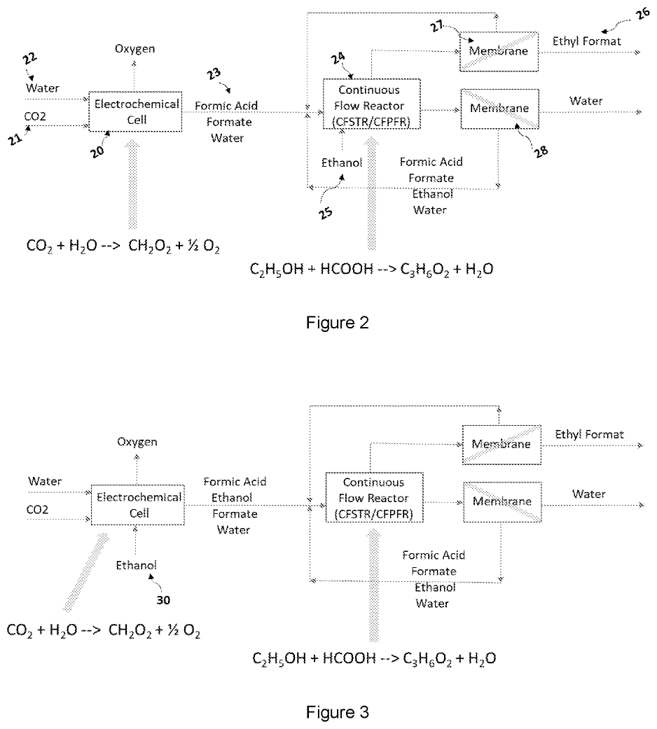

An embodiment of the present invention is shown in FIG. 2, which illustrates the conversion of CO₂ to ethyl formate using a continuous flow reactor such as continuous flow stirred tank reactor (CFSTR) or continuous flow plug flow reactor (CFPFR) with low GHG membrane separation processes. Electrochemical cell (20) uses CO₂ (21) and water (22) as feed. At the anode electrode in the electrochemical cell water is split into protons and electrons, and oxygen is generated. At the cathode electrode CO₂ reacts with the protons and the electrons to form formate ions.

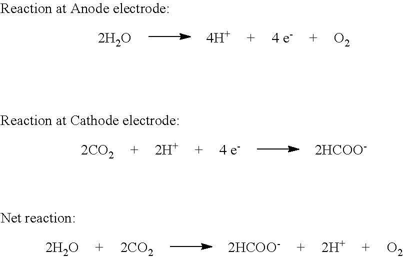

CO₂ (21) feed for the electrochemical cell (20) is preferably substantially free of ionic impurities, which may interfere in the electrochemical reaction. Water (22) used also is preferably substantially ion free (deionized water). In a preferred embodiment the H₂ is produced by electrolysis, however, it can be supplied by any number of sources known to those skilled in the art, including “green” hydrogen and “blue” hydrogen sources. Electrochemical cells (20) for this conversion are available in various configurations usable in this invention. The formic acid produced in the electrochemical cell is in dilute aqueous state that is about 10 wt %.

Formic acid in conventional industrial process is also produced in dilute aqueous state. On industrial scale it is conventionally produced by carbonylation of methanol with carbon monoxide to first make methyl formate.

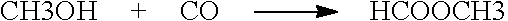

In the second stage methyl formate is hydrolyzed to formic acid and methanol.

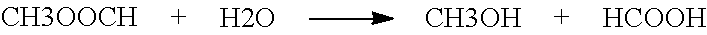

Since formic acid catalyzes esterification, at equilibrium all four components, methyl formate, water, methanol and formic acid are present in high proportions. Equilibrium is shifted towards formic acid by excess of water resulting in the production of aqueous formic acid.

The formic acid (23) in aqueous media, is processed in a continuous flow reactor (“CFSTR” or “CFPFR”) (24) where it reacts with ethanol (25) to produce ethyl formate (26).

Esterification reaction kinetics with ethanol has been reported but not in a highly dilute (˜10 wt % acid) aqueous media. This difference was one of the challenges to overcome in the present invention. In this innovative process we have eliminated conventional energy-intensive separation techniques such as distillation/extractive-distillation which are required to produce a higher concentration of formic acid as a raw material, as well as to produce ethyl formate at commercial grade purity.

The substantially continuous removal of ethyl formate, as it forms, using a membrane system (27) connected and being integral part of the continuous flow reactor (24), as taught herein, is one feature of the present invention as shown in FIG. 2. Ethyl formate (26), a low boiler compared to other components in the reactor, with significantly higher vapor pressure, is separated from the reaction mixture using membrane pervaporation process. This produces higher purity ethyl formate (26) as well as assist in achieving higher conversion of formic acid to ethyl formate. The remaining stream from the membrane separation system (27) is recycled back to the continuous flow reactor (24). Another membrane system (28), shown in FIG. 2, is used to remove excess water formed during esterification reaction in order to avoid continuous rise in water concentration in the continuous flow reactor (24).

There are several membranes which can be used for the separation of ethyl formate from the reaction mixture which includes formic acid, ethanol and water. Hydrophobic membranes such as made of polydimethylsiloxane, polytetrafluoroethylene and others are potential candidates for permeating ethyl formate in pervaporation mode over other reaction mixture components. In addition, a liquid membrane such as a membrane containing non-volatile liquid in pores may be suitable for permeation of ethyl formate from the reaction mixture. Zeolite membranes, due to their precise molecular sieving properties and the ability to selectively adsorb or exclude molecules based on their size and polarity could also be used for selective separation of ethyl formate from reaction mixture. Zeolite membranes consist of crystalline microporous materials with uniform pore sizes, allowing them to act as molecular sieves. Zeolites can be tailored during synthesis to achieve specific adsorption properties, making them suitable for separating molecules with subtle differences.

For water separation from the reaction mixture polymeric membranes such as nafion, a perfluorinated polymer membrane with high proton conductivity and high selectivity for water, may be used herein. It can effectively separate water from organic molecules due to its preferential permeation of water molecules. Polyamide is another membrane that can be used for selective separation of water from other organic molecules. Thin-film composite membranes based on polyamide selective layers may also be used. Ceramic membranes such as alumina (Al₂O₃) and zirconia (ZrO₂), having chemical resistance and thermal stability, can also selectively permeate water while rejecting organic molecules based on differences in molecular size and polarity. Composite membranes that combine a polymer matrix with ceramic nanoparticles or layers can provide enhanced selectivity and mechanical strength, can also be used. lon-exchange membranes such as sulfonated polymeric membranes contain sulfonic acid groups that can selectively transport protons (H+) or water molecules, can also exhibit high selectivity for water over organic molecules due to preferential proton transport. Graphene oxide membranes exhibit high water permeability and selectivity due to the two-dimensional structure of graphene sheets. They may effectively separate water from organic molecules based on differences in molecule size and interactions with the graphene surface

In another embodiment of this inventive process, ethanol (30) can be directly added to the electrochemical cell as shown in FIG. 3. This illustrates CO₂ conversion to ethyl formate with ethanol added to the electrochemical cell.

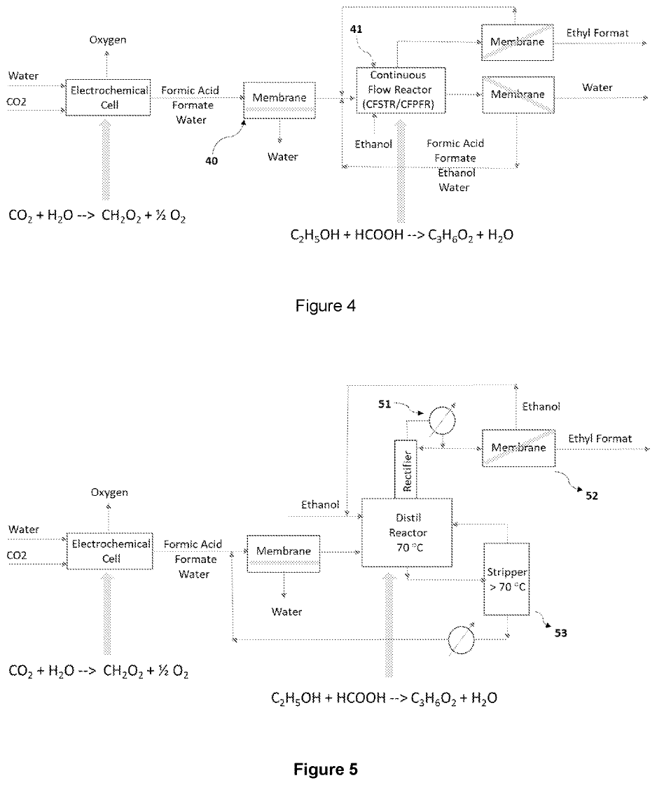

FIG. 4 illustrates another aspect of this innovative process for conversion of CO₂ to ethyl formate utilizing a membrane to remove water from electrochemical product slate to improve formic acid concentration to the continuous flow reactor. A membrane system (40) is used to reduce water in the product slate coming out of the electrochemical cell. This process improves the formic acid concentration to the continuous flow reactor (41) and proportionally reduces the concentration of ionized formate. This arrangement improves conversion of formic acid to ethyl formate. The membranes described above for water separation from reaction mixture may also be used for water separation from formic acid.

In another embodiment, the esterification reaction can be carried out at about 70° C. This vaporizes the product, ethyl formate, with some other components such as ethanol from the reactor. The vapors are condensed in a condenser (51) as shown in FIG. 5. Ethyl formate forms azeotrope with ethanol. The membrane (52) separation operated in pervaporation mode can separate ethyl formate from ethanol and other components. Ethyl formate will be collected as a product and rest to be recycled back to the reactor. The reactor effluent to be processed in a stripper (53) with lower boiler ethanol and remaining ethyl formate as overhead going back to the reactor. The stripper bottom, rich in water, is recycled back to the membrane system removing water from the feed stream from the electrochemical cell.

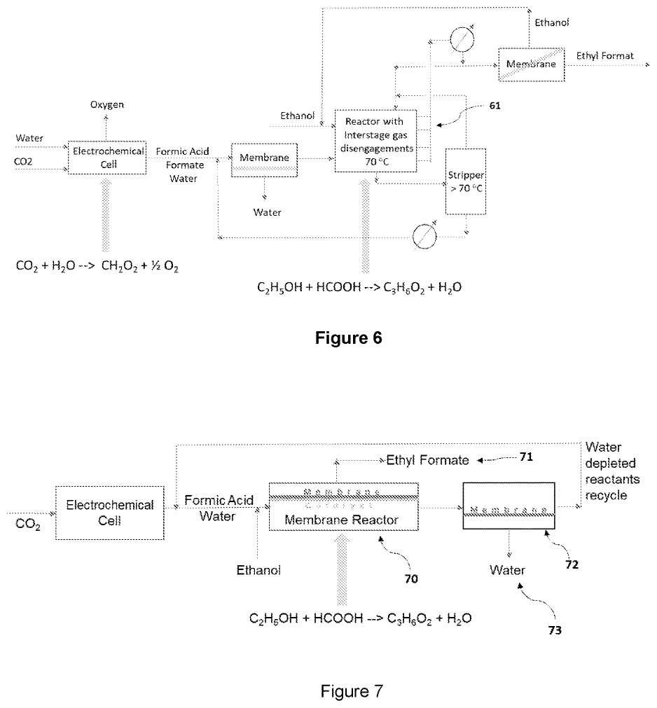

In another embodiment, shown in FIG. 6, the continuous flow reactor can be operated in multiple stages (61) with vapor recovery from each stage and combining them together as one stream going to the overhead condenser.

In another aspect of this innovative process, the continuous flow reactor can be replaced by a membrane reactor (70) as shown in FIG. 7. In the membrane reactor (70) ethyl formate (71) can be continuously removed by the membrane operating in pervaporation mode. This produces a reaction equilibrium shift to higher conversion of formic acid to ethyl formate. The remaining stream exiting the membrane reactor, containing unreacted non-ionized formic acid, ionized formate, and ethanol, will be recycled back to the membrane reactor. This stream will have a higher concentration of water as syntheses of ethyl formate produce water. A membrane system (72) will be used to remove excess water (73), before recycling the stream, to facilitate matching or exceeding the formic acid concentration in the stream to the fresh feed to the reactor.

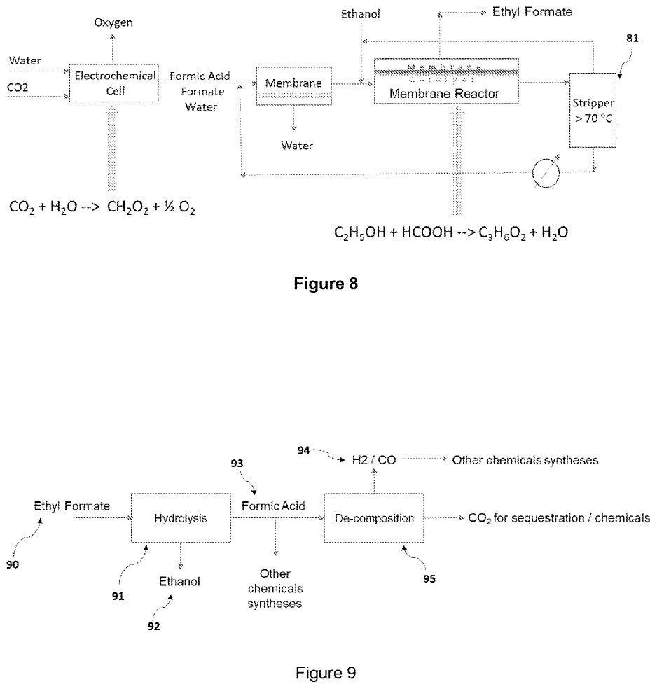

In another embodiment membrane reactor effluent stream is processed in a stripper (81), as shown in FIG. 8. The overhead of the stripper richer in ethanol is recycled back to the membrane reactor inlet. The stripper bottom is recycled back to the water removal membrane system separating water from the feed stream to the membrane reactor.

In addition to forming ethyl formate as a final commodity chemical product, the present invention may be used for producing useful chemical intermediates. FIG. 9 illustrates Ethyl formate (90) hydrolyzed (91) to form concentrated ethanol (92) and formic acid (93). Formic acid can be used alone for certain chemical syntheses, e.g., as an olefins hydroformulation reagent. If desired, as shown in FIG. 9, the resulting formic acid can be used as a hydrogen and/or CO carrier (94) by decomposing (95) the formic acid. Since this decomposition is more thermodynamically favorable than hydrolysis with appropriate catalysis these steps may be combined for efficiency gains.

The following non-limiting example serves to illustrate the proposed process invention.

### EXAMPLE 1

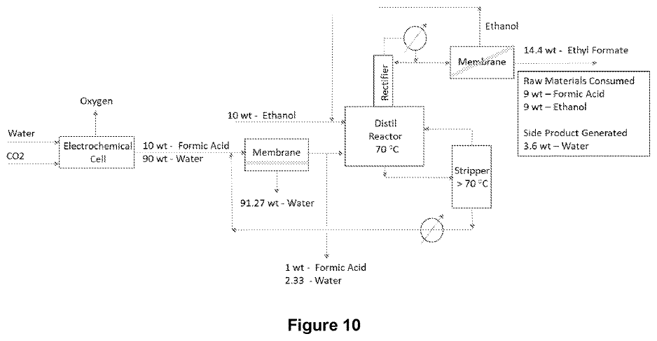

A 10 wt % formic acid in water stream, generated in an electrochemical cell process, is fed to a membrane unit as shown in FIG. 10. For materials balance perspective, it is assumed that the total feed is 100 wt (any weight unit) containing 10 wt % formic acid and 90 wt % water. The simulation run on the process shown in FIG. 10 mixes the fresh feed from the electrochemical cell with the recycle stream from the stripper bottom before processing in the membrane unit to remove 91.27 wt water. The stream coming out of the membrane process after water removal has 30 wt % formic acid in water. It has increased the formic acid concentration from 10 wt % to 30 wt %. Part of this stream containing 1 wt formic acid and 2.33 wt of water is purged and the rest is fed to the reactor which is operating at about 70° C. Ethyl formate generated in the reactor evaporates from the reactor along with other components, mainly ethanol. The vapor is condensed in a condenser and the condensed liquid is processed through a pervaporation membrane unit to produce 14.4 wt of ethyl formate. The remaining stream containing mainly ethanol is recycled back to the reactor. 14.4 wt ethyl formate produced corresponds to formic acid and ethanol consumption of 9 wt each. It also corresponds to esterification reaction water generation of 3.6 wt. This shows a 90% conversion of formic acid to ethyl formate. Process parameters may be adjusted to vary the conversion, i.e., ethyl formate yield.

1. A method for conversion of CO₂ to ethyl formate comprising:
a) Reducing CO₂ in the presence of water in an electrochemical cell, to form formate and hydroxide ions,
b) Reacting the formate and hydroxide ions with hydrogen ions to form formic acid and water,
c) Processing the formic acid and water in a reactor with ethanol to produce a substantially continuous product stream containing ethyl formate.

2. The method of claim 1 wherein the CO₂ is substantially free from ionic impurities.

3. The method of claim 1 or 2 wherein the water is substantially ion free.

4. The method of claim 1 wherein the hydrogen ions are supplied by green hydrogen, blue hydrogen, or a combination thereof.

5. The method of claim 1 wherein the electrochemical cell operating parameters maintain a product ratio of non-ionized formic acid to formate ion of greater than about 1.

6. The method of claim 1 wherein the reactor is a continuous flow reactor.

7. The method of claim 6 wherein the reactor is a continuous flow plug flow reactor.

8. The method of claim 6 wherein the reactor is a continuous flow stirred tank reactor.

9. The method of claim 6 wherein at least a portion of the ethyl formate is removed from the reactor as formed.

10. The method of claim 9 wherein a membrane system is used to remove the ethyl formate in the reactor.

11. The method of claim 10 wherein the membrane is a pervaporation membrane.

12. The method of claim 11 wherein the product stream remaining after the removal of ethyl formate is recycled to the continuous flow reactor.

13. The method of claim 12 wherein the membrane or membranes separate water whereby the remaining formic acid recycled to the continuous flow reactor has a concentration of greater than about ten percent.

14. The method of claim 13 wherein the formic acid concentration is greater than about twenty five percent.

15. The method of claim 14 wherein the formic acid concentration is greater than about fifty percent.

16. The method of claim 15 wherein ethanol is added to the electrochemical cell.

17. The method of claim 16 wherein the reaction in the reactor is conducted at a temperature of about seventy degrees C.

18. A method for conversion of dilute formic acid plus ethanol to ethyl formate comprising processing formic acid and water at ratio of non-ionized formic acid to formate ion of greater than about one, in a continuous flow reactor to produce a product stream containing ethyl formate.

19. The method of claim 18 wherein at least a portion of the ethyl formate is removed from the reactor as formed.

20. The method of claim 19 wherein a membrane system is used to remove the ethyl formate from the reactor in a substantially continuous process.

21. The method of claim 20 wherein the membrane is a pervaporation membrane.

22. The method of claim 21 wherein the product stream remaining after the removal of ethyl formate is recycled back to the reactor using a membrane that removes at least a portion of water.

23. The method of claim 22 wherein the membrane is a pervaporation membrane.
